(19) United States
(12) Patent Application Publication
(10) Pub. No.: US 2010/0179156 A1
(43) Pub. Date: Jul. 15, 2010

(54) Antimicrobial compositions

(76) Inventors: Robert Engel, Carle Place, NY (US); JaimeLee Iolani Rizzo, Glen Cove, NY (US); Karin Melkonian Fincher, Garden City, NY (US)

Correspondence Address: HOFFMANN & BARON, LLP, 6900 JERICHO TURNPIKE, SYOSSET, NY, 11791, US

(21) Appl. No.: 12/310,636

(22) PCT Filed: Aug. 28, 2007
(86) PCT No.: PCT/US07/18882

§ 371 Date: Feb. 27, 2009

Related U.S. Application Data
(60) Provisional application No. 60/823,960, filed on Aug. 30, 2006.
(60) Provisional application No. 60/863,147, filed on Oct. 27, 2006.

(51) Int. Cl. A01N 43/60; C07D 487/08; A01P 1/00

(52) U.S. Cl. 514/249; 544/351

(57) ABSTRACT

The present invention is directed to providing antimicrobial surfaces containing 1,4-diazoniabicyclo[2.2.2]octane and hydrocarbon groups and/or chains. More specifically, the present invention is directed to antimicrobial compositions wherein the hydroxyl groups on polyols are replaced by 1,4-diazoniabicyclo[2.2.2]octane. The invention is also directed to methods of making antimicrobial compositions containing polyols.

### CROSS-REFERENCE TO RELATED APPLICATION

This application claims the benefit of U.S. Provisional Application No. 60/823,960, filed Aug. 30, 2006, and U.S. Provisional Application No. 60/863,147, filed Oct. 27, 2006, which are incorporated herein by reference.

### BACKGROUND OF THE INVENTION

Current fears of antibiotic-resistant bacteria and other microbes as well as of bioterrorism have increased the importance of developing new ways to protect people from microbial infection. It is, for example, important to develop new compositions that can be applied to a surface to provide antimicrobial protection without creating antibacterial resistant microbes. Such compositions would be useful, for example, in hospitals and during military and civilian operations where bacterial contamination has occurred, or is expected to occur.

In developing new antimicrobial compositions, it is important to discourage further antibiotic resistance. Ideally, therefore, novel antimicrobial compositions will function through non-specific, non-metabolic mechanisms.

For example, polycationic (quaternary ammonium) strings were developed in the laboratory of Robert Engel. See Fabian et al, Syn. Lett., 1007 (1997); Strekas et al, Arch. Biochem. and Biophys. 364, 129-131 (1999). These strings are reported to have antibacterial activity. See Cohen et al, Heteroat. Chem. 11, 546-555 (2000).

There is, clearly, a need for improved new antimicrobial compositions that can be easily applied to surfaces, e.g., skin. Ideally, the antimicrobial compositions do not lead to bacterial resistance.

### SUMMARY OF THE INVENTION

These and other objectives will be apparent to those having ordinary skill in the art have been achieved by providing an antimicrobial composition comprising a carrier and a chemical compound having the following formula (1):

R¹—Y¹—X—Y²—R²

wherein:
X represents 1,4-diazoniabicyclo[2.2.2]octane;
Y¹ and Y² independently represent hydrocarbon chains comprising a minimum of 10 carbon atoms and a maximum of 24 carbon atoms;
R¹ and R² independently represent H, halo, or OR³;
R³ represents H or R⁴;
R⁴ represents —C(O)R⁵ or R⁶;
R⁵ represents H or a hydrocarbon group comprising a minimum of 1 carbon atom and a maximum of 4 carbon atoms; and
R⁶ represents a hydrocarbon group comprising a minimum of 1 carbon atom and a maximum of 4 carbon atoms.

In another embodiment, the invention relates to an antimicrobial composition comprising a chemical compound having the following formula (2):

R¹—Y¹—X—Z—(X—Y²—R²)ₙ

wherein:
Z represents a polyol having more than one primary hydroxyl group wherein at least two of the primary hydroxyl groups have been replaced by R¹—Y¹—X or R²—Y²—X groups;
X represents 1,4-diazoniabicyclo[2.2.2]octane;
Y¹ and Y² independently represent hydrocarbon chains comprising a minimum of 10 carbon atoms and a maximum of 24 carbon atoms;
R¹ and R² independently represent H, halo, or OR³;
R³ represents H or R⁴;
R⁴ represents —C(O)R⁵ or R⁶;
R⁵ represents H or a hydrocarbon group comprising a minimum of 1 carbon atom and a maximum of 4 carbon atoms;
R⁶ represents a hydrocarbon group comprising a minimum of 1 carbon atom and a maximum of 4 carbon atoms; and
n represents any number up to m−1 wherein m represents the number of primary hydroxyl groups in the polyol.

In yet another embodiment, the invention relates to a method of making an antimicrobial composition comprising a chemical compound having the following formula (2):

R¹—Y¹—X—Z—(X—Y²—R²)ₙ

wherein:
Z represents a polyol having more than one primary hydroxyl group wherein at least two of the primary hydroxyl groups have been replaced by R¹—Y¹—X or R²—Y²—X groups;
X represents 1,4-diazoniabicyclo[2.2.2]octane;
Y¹ and Y² independently represent hydrocarbon chains comprising a minimum of 10 carbon atoms and a maximum of 24 carbon atoms;
R¹ and R² independently represent H, halo, or OR³;
R³ represents H or R⁴;
R⁴ represents —C(O)R⁵ or R⁶;
R⁵ represents H or a hydrocarbon group comprising a minimum of 1 carbon atom and a maximum of 4 carbon atoms;
R⁶ represents a hydrocarbon group comprising a minimum of 1 carbon atom and a maximum of 4 carbon atoms; and
n represents any number up to m−1 wherein m represents the number of primary hydroxyl groups in the polyol,
   the method comprising:
(a) providing a solution of a polyol;
(b) converting at least two primary hydroxyl groups of the polyol to leaving groups; and
(c) adding R—Y-Q,
   wherein R represents H, halo, or OR³;
   Y represents a hydrocarbon chain comprising a minimum of 10 carbon atoms and a maximum of 24 carbon atoms; and
   Q represents 1-azonia-4-azabicyclo[2.2.2]octane.

### DETAILED DESCRIPTION OF THE INVENTION

The invention relates to novel antimicrobial compositions suitable for protecting surfaces and compositions from microbial (e.g., bacterial) infestation. Any surface on which microbes can survive and grow can be treated with the antimicrobial compositions of the invention. Some examples include the surfaces of metals, wood, plastic, glass, protein and carbohydrate. The surfaces can be those of medical devices and instruments; athletic clothing and equipment; synthetic materials, such as polyester and rayon; and food, such as vegetables, tubers, fruit and the like.

Similarly, any composition in which microbes can survive and grow can be treated with the antimicrobial compositions of the invention. Some examples of compositions include paint, toothpaste, lotions, and cosmetics.

In this specification, a distinction is made between hydrocarbon groups and hydrocarbon chains. A hydrocarbon group is bonded at only one end to another chemical moiety. A hydrocarbon chain is bonded independently at each end to another chemical moiety, e.g., to a group, or to an atom.

### Antimicrobial Compounds

In one aspect of the invention, the compositions comprise antimicrobial compounds having the following structure:

R¹—Y¹—X—Y²—R².

### Formula 1

In formula 1, X represents 1,4-diazoniabicyclo[2.2.2]octane, as shown below.

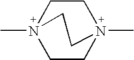

Y¹ and Y² independently represent hydrocarbon chains. R¹ and R² independently represent H, halo, or OR³, wherein halo means fluoro, chloro, bromo, or iodo; R³ represents H or R⁴; R⁴ represents —C(O)R⁵ or R⁶; R⁵ represents H or a hydrocarbon group; and R⁶ represents a hydrocarbon group.

The hydrocarbon groups of R⁵ and R⁶ comprise a minimum of one carbon atom and a maximum of four carbon atoms (i.e., C₁-C₄). The carbon atoms of a group can all be saturated, or can all be unsaturated. Alternatively, the group can comprise a mixture of saturated and unsaturated carbon atoms. The unsaturated hydrocarbon groups contain one or more double and/or triple bonds.

Some examples of hydrocarbon groups include methyl, ethyl, propyl, propenyl, isopropyl, butyl, t-butyl, s-butyl, and 1- or 2-butynyl. Additional hydrocarbon groups include 3-butenyl and 1,3-butadienyl. The preferred hydrocarbon groups are methyl and ethyl.

Hydrocarbon chains in formula 1 are unbranched. The carbon atoms of a chain can all be saturated, or can all be unsaturated. Alternatively, a chain can comprise a mixture of saturated and unsaturated carbon atoms. The unsaturated hydrocarbon chains contain one or more double and/or triple bonds.

The minimum number of carbon atoms in a hydrocarbon chain of Y¹ and Y² is 10. The maximum number of carbon atoms in a hydrocarbon chain of Y¹ and Y² is 24. Preferred chain lengths for Y¹ and Y² are 12 or 16 carbon atoms. In one illustrative embodiment, Y¹ represents a hydrocarbon chain of 12 carbon atoms and Y² represents a hydrocarbon chain of 16 carbon atoms. In another embodiment, Y¹ and Y² both represent 12 carbon atoms. In yet another embodiment, Y¹ and Y² both represent 16 carbon atoms.

Some examples of saturated C₁₀-C₂₄ hydrocarbon chains include decyl, dodecyl, tetradecyl, hexadecyl, and octadecyl chains. Some examples of unsaturated C₁₀-C₂₄ hydrocarbon chains include oleyl, linoleyl, and linolenyl, especially cis-oleyl, cis,cis-linoleyl, and cis, cis, cis-linolenyl chains.

In another aspect of the invention, the compositions comprise antimicrobial compounds having the following structure:

R¹—Y¹—X—Z—(X—Y²—R²)ₙ.

### Formula 2

In formula 2, R¹, R², Y¹, Y² and X represent the same structures with the same limitations, properties, and/or preferences as described above for the compounds having formula 1.

The antimicrobial compounds of the invention, e.g., Formula 1 or Formula 2, contain one or more anions to balance the charge of the quaternary ammonium groups. The anion may be singly charged, doubly charged, or triply charged. Some examples of anions include monovalent anions such as halides (e.g., F⁻, Cl⁻, Br⁻, and I⁻), OH⁻, and H⁻ divalent anions such as S⁻², CO₃⁻², SO₄⁻², and trivalent anions such as PO₄⁻³, and PO₃⁻³.

### Polyols

Z, in formula 2, represents a polyol, i.e., a modified polyol, having more than one primary hydroxyl group in its unmodified state, wherein at least two of the primary hydroxyl groups in the unmodified state have been replaced by R¹—Y¹—X or R²—Y²—X groups. The polyol, i.e., the unmodified polyol, can be any molecule having more than one primary hydroxyl group. The unmodified polyol may, for example, be an alkane polyol, a polyether, a carbohydrate, or a protein.

An alkane polyol of the present invention is an alkane with a minimum of two carbon atoms and a maximum of twelve carbon atoms, and at least two primary hydroxyl groups. Some examples of alkane polyols include glycerol; mannitol; ethylene glycol; 1,5-pentanediol; 1,2,3,4,5,6,7,8-octaneoctol; 1,6,12-dodecanetriol; and 3-methanolyl-1,6-hexanehexyl.

The polyol, e.g. the unmodified polyol, can be a polyether. In this specification, polyethers refer to molecules with more than one ether group and at least two primary hydroxyl groups, e.g. the polyether may have a minimum of one ether group, and a maximum of about 10,000, preferably about 1,000, more preferably about 100, and most preferably about 10 ether groups. Some examples of polyethers include polyethylene glycol and polytetrahydrofuran (i.e., poly(tetramethylene ether glycol, OH(OCH₂CH₂CH₂CH₂)ₙOH)).

Carbohydrates include saccharides, e.g., monosaccharides, oligosaccharides, and polysaccharides. The minimum number of saccharide units in an oligosaccharide is two. The maximum number of saccharide units in an oligosaccharide is typically twelve, preferably ten.

Polysaccharides have more than twelve saccharide units, and may have up to several thousand units, e.g. up to a maximum of about 10,000. In this specification, polysaccharides refer to polymers of (+)-glucose, and include cellulose, starch and glycogen. The saccharides can be in either the D or L configuration. Saccharide units can be either aldoses or ketoses.

The number of carbons of a saccharide unit can be from three carbons to about six carbons. An example of a three carbon sugar is glyceraldehyde. Examples of four carbon sugars include erythrose and threose. Examples of five carbon sugars include ribose, arabinose, xylose and lyxose. Examples of six carbon sugars include allose, altrose, glucose, mannose, gulose, idose, galactose and talose. All of these saccharides further include the corresponding 2′-deoxy derivatives.

The polyol can be a polyamino acid having at least two amino acids with primary hydroxyl groups. Polyamino acids include oligopeptides and proteins. An oligopeptide has two to twelve amino acid residues. Typically, proteins have more than twelve amino acid residues and up to about 1,000 amino acid residues.

The letter n in formula 2 represents any number up to m−1 wherein m represents the number of primary hydroxyl groups in the polyol, Z, i.e., the unmodified polyol. For example, n represents the number of hydroxyl groups that have been replaced by R¹—Y¹—X or R²—Y²—X, and may be any number greater than zero and up to m−1. The minimum values for m are two, four, and six. The maximum number for m depends upon the type of polyol.

Carbohydrates can contain several thousand saccharide units. Each saccharide unit typically contains one primary hydroxyl group. Typically, for a carbohydrate, m should not be greater than 10,000.

Proteins can contain up to 1,000 amino acid residues and sometimes more. A typical protein contains about 300 amino acid residues. Of the twenty naturally occurring amino acids, only serine contains a primary hydroxyl group. Typically, m should not be greater than 200 for a protein.

Preferably, the alkane polyols of the present invention contain a minimum of two carbon atoms and a maximum of twelve carbon atoms, and at least two primary hydroxyl groups. Typically, m should not be greater than eight for an alkane polyol of the present invention.

For example, when Z is 2,3-hydroxymethyl-1,4-butanediol, the alkane polyol contains four primary hydroxyl groups. The value of m is 4 and n may be any number up to 3. An antimicrobial composition for 2,3-hydroxymethyl-1,4-butanediol may, for instance, have a value for n of 2.

In a preferred embodiment, the polyol is a gelling agent. Some examples of gelling agents include polysaccharides, proteins, and mixtures thereof.

An example of carbohydrate gelling agents are gums. An example of a natural gum is locust bean gum.

Another example of a polysaccharide gelling agent is a pectin, agar, alginic acid or carrageenan, or a salt thereof. Some examples of salts of alginic acid include sodium alginate, potassium alginate, ammonium alginate and calcium alginate.

Protein gelling agents include gelatin. Some examples of protein gelatin agents include gelatin A, gelatin B, and collagen.

An advantage of the compounds of formula 2 where Z is a gelling agent is that they comprise an internal gelling agent covalently bonded to an antimicrobial 1,4-diazabicyclo[2.2.2]octane or 1-azonia-4-azabicyclo[2.2.2]octane moiety. Accordingly, the compounds are able to form gels without addition of further gelling agents. A preferred composition is a gel that comprises a compound according to formula 2 where Z is a gelling agent in the absence of a further gelling agent, e.g., a gel that consists essentially of the chemical compound and water.

### Activation of Hydroxyl Groups

Hydroxyl groups in the compounds useful in the compositions of the invention (e.g., polyols) can be activated for covalent bonding to 1,4-diazabicyclo[2.2.2]octane or 1-azonia-4-azabicyclo[2.2.2]octane by methods known in the art. Activation of hydroxyl groups may be accomplished by converting the hydroxyl groups to electrophilic leaving groups. Suitable electrophilic leaving groups include, for example, a halo group or an active ester group.

Some suitable halo groups include chloro and bromo. Hydroxyl groups may, for example, be converted to chloro or bromo groups by treatment with thionyl chloride or phosphorus tribromide, respectively.

Suitable ester leaving groups include sulfonic acid esters. Hydroxyl groups may be converted to sulfonic acid esters by treating the hydroxyl groups with a reagent in a suitable medium. The reagent may, for example, include benzenesulfonyl chloride, p-toluenesulfonyl chloride, and methanesulfonyl chloride. Suitable media for the reaction include, but are not limited to, pyridine, hexane, heptane, ether, toluene, ethyl acetate, and mixtures thereof.

The amount of reagent, volume of suitable medium, and other reaction conditions are known to those in the art.

The activated polyols are then treated with 1,4-diazabicyclo[2.2.2]octane or with (formula 3) wherein Q represents 1-azonia-4-azabicyclo[2.2.2]octane under conditions that cause the leaving groups to be replaced. R¹ and Y¹ are as described above. Such conditions are well known in the art.

It is not necessary to activate all of the available primary hydroxyl sites present on the surface of a material. For example, at least about 10% of the available hydroxyl groups on a surface may be activated to subsequently provide sufficient antimicrobial activity. Preferably, at least about 25% of the available hydroxyl groups may be activated, more preferably at least about 50%, and most preferably at least about 75% of the available hydroxyl groups may be activated.

For example, when Z is a carbohydrate comprising 2,000 glucose units, m is 2,000, and n may be any number up to 1,999. An antimicrobial composition for a 2,000 unit carbohydrate may, for instance, have a value for n of 1,500.

In another example, when Z is a protein comprising 300 amino acid residues, fifteen of which are serine, m is fifteen, and n may be any number up to fourteen. An antimicrobial composition for a 300 residue protein may, for instance, have a value for n of seven.

### Pharmaceutical Compositions

The compositions of the present invention comprise compounds of formula 1 or formula 2 and suitable carriers (e.g., pharmaceutical acceptable carriers) for topical application. The compositions can be in any form as would be known by a skilled artisan. For example, the compositions can be in the form of a lotion, spray, or paste. In the lotion form, the compounds are part of a pourable emulsion of oil and water. In the spray form, the compounds are dispersed as a liquid in a gas in which liquid droplets have diameters greater than 10 micrometers. In the paste form, the compounds are suspended in a viscous fluid. Topical application of the compositions in amounts of up to about 25% (w/w) in a carrier are suitable. The amounts can be adjusted according to the purpose of the composition as would be known by a skilled artisan.

Alternatively, the compound according to formula 1 or formula 2 can be combined with a gelling agent to form a gel. Some examples of gelling agents, including pharmaceutically acceptable gelling agents, include gums, especially natural gums, starches, pectins, agar and gelatin.

The pharmaceutical compositions of the present invention can contain the compounds of formula 1 or formula 2 where Y¹ and Y² each represents a mixture of different hydrocarbon chains. In a preferred embodiment, the pharmaceutical compositions comprise compounds in which at least about 25%, preferably at least about 50%, more preferably at least about 75%, and most preferably at least about 90%, of the hydrocarbon chains of Y¹ and Y² have 12 carbon atoms and 16 carbon atoms, respectively.

In a preferred embodiment, the invention relates to novel pharmaceutical compositions suitable for topical administration. The compositions have antimicrobial activity, and can be easily prepared and applied to human surfaces. Such surfaces include, for example, skin, as well as surfaces of the mucosa that are accessible to topical administration, for example, buccal, intranasal, anal, and vaginal surfaces.

### Antimicrobial Activity

The compositions that include the antimicrobial compounds according to the invention demonstrate excellent antimicrobial properties. In this specification, antimicrobial properties refer to the ability to resist growth of single cell organisms, e.g., bacteria, fungi, algae, and yeast, as well as mold.

The bacteria include both gram positive and gram negative bacteria. Some examples of Gram positive bacteria include, for example, Bacillus cereus, Micrococcus luteus, and Staphylococus aureus. Some examples of Gram negative bacteria include, for example, Escherichia coli, Enterobacter aerogenes, Enterobacter cloacae, and Proteus vulgaris. Strains of yeast include, for example, Saccharomyces cerevisiae.

A particular advantage of such action is the lack of consumption of the antimicrobial agent. Moreover, the antimicrobial activity is non-specific and non-metabolic. Therefore, the danger of encouraging resistant strains of microbes is reduced.

In order to demonstrate the antimicrobial properties achieved in accordance with the invention, the compositions of the invention were applied to different surfaces and tested for antimicrobial activity. The results are described below.

### EXAMPLES

In the examples below, terms such as dabco-Cn refer to compounds having the formula R¹—Y¹-Q, i.e., formula 3 above, wherein R¹ represents H, Y¹ represents a hydrocarbon chain with n carbon atoms, and Q represents 1-azonia-4-azabicyclo[2.2.2]octane. Thus, dabco-C12 means 1-dodecyl-1-azonia-4-azabicyclo[2.2.2]octane.

### Example 1a

### Preparation of Gelatin B-dabco-C12, 14, 16

Thirty grams of Gelatin B is dissolved in 1 equivalent of tosyl chloride (TsCl) in a saturated sodium bicarbonate solution. The solution is allowed to react for a day at room temperature. Tosylated Gelatin B is washed with water and dried. Tosylated Gelatin B is then added to a one mol equivalent solution of dabco-Cn (n=a mixture of 12, 14, and 16) in water and allowed to react for three days. The solid Gelatin B product is washed with water and allowed to dry.

### Example 1b

### Preparation of Gelatin A-dabco-C12, Gelatin A-dabco-C14, and Gelatin A-dabco-C16

Thirty grams of Gelatin A is dissolved in 1 equivalent of TsCl in a saturated sodium bicarbonate solution. The solution is allowed to react for a day at room temperature. Tosylated Gelatin A is washed with water and dried. Tosylated Gelatin A is then added to a one mol equivalent solution of dabco-Cn (n=12, 14, or 16) in water and allowed to react for three days. The solid Gelatin A product is washed with water and allowed to dry.

### Example 2

### Preparation of Agarose-OTS

Five grams of Agarose is treated with 1 mol equivalent of TsCl (5.33 grams). The Agarose/TsCl mixture is added to a saturated NaHCO₃ solution. The NaHCO₃ mixture is stirred for 3 days at room temperature.

### Example 3

### Preparation of Agarose-OTS

Ten grams of Agarose is treated with 1 mol equivalent of TsCl (10.7 grams). The Agarose/TsCl mixture is added to a saturated NaHCO₃ solution. The NaHCO₃ mixture is stirred for 1 day at room temperature.

### Example 4

### Preparation of Blue Paper-Agarose-OTS (Unwashed)

Six strips of Blue paper are added to Agarose-OTS, prepared according to example 3, for 5 minutes. The strips are then air dried (unwashed) for use as a control group.

### Example 5

### Preparation of Agarose-dabco-C16

32.27 grams of Agarose-OTS prepared according to Example 2 is treated with 1 mol equivalent of dabco-C16 (38.25 grams) in H₂O. The treated mixture is centrifuged for 7 minutes at 100,000 rmp to remove excess H₂O. The remaining solution is stirred at room temperature for 3 days.

### Example 6

### Preparation of Blue Paper-Agarose-OTS (Washed)

Eight strips of Blue Paper are added to the Agarose-OTS prepared according to Example 3 and stirred at room temperature for 5 minutes. The strips are washed in tap water for 5 minutes, and then air dried.

### Example 7

### Preparation of Microscope Glass-Agarose-OTS (Washed)

Three microscope glass slides are added to the Agarose-OTS prepared according to Example 3 and stirred at room temperature for 3 days. The slides are washed in tap water for 5 minutes and then air dried. The resulting slides are used as a control group.

### Example 8

### Preparation of Microscope Glass-Agarose-OTS (Unwashed)

Three microscope glass slides are added to Agarose-OTS prepared according to Example 3 at room temperature 3 days. The slides are air dried. The resulting slides are used for as a control group.

### Example 9

### Preparation of Microscope Glass-Agarose-dabco-C16 (washed)

Three microscope glass slides are added to Agarose-OTS prepared according to Example 3 and stirred at room temperature for 3 days. The slides are then added to dabco-C16 and stirred at room temperature for 1 day. The slides are then washed for 5 minutes in tap water and air dried.

### Example 10

### Preparation of Microscope Glass-Agarose-dabco (Unwashed)

Three microscope glass slides are added to Agarose-OTS prepared according to Example 3 and stirred at room temperature for 3 days. The slides are then added to dabco-C16 and stirred at room temperature for 1 day. The slides are then air dried.

### Example 11

### Preparation of Plastic Strips-Agarose-OTS (Washed)

Three plastic strips are added to Agarose-OTS prepare according to Example 2 and stirred at room temperature for 1 day. The strips are then washed for 5 minutes in tap water and then air dried.

### Example 12

### Preparation of Plastic Strips-Agarose-OTS (Unwashed)

Three plastic strips are added to Agarose-OTS prepared according to Example 2 and stirred at room temperature for 1 day. The strips are then air dried. The strips can be used as a control group.

### Example 13

### Preparation of Plastic Strips-Agarose-dabco-C16 (washed)

Three plastic strips are added to Agarose-OTS prepared according to Example 2 and stirred at room temperature for 1 day. The strips are then added to dabco-C16 and stirred at room temperature for 1 day. The strips are then washed for 5 minutes in tap water and air dried.

### Example 14

### Preparation of plastic Strips-Agarose-dabco-C16 (unwashed)

Three plastic strips are added to Agarose-OTS prepared according to Example 2 and stirred at room temperature for 1 day. The strips are then added to dabco-C16 and stirred at room temperature for 1 day. The strips are then air dried.

### Example 15

### Preparation of Agarose-OTS

Five grams of Agarose is treated with 1 mol equivalent of TsCL (5.325 g) in a saturated NaHCO₃ solution and stirred at room temperature for 2 hours.

### Example 16

### Preparation of Microscope Glass-Agarose-OTS

Ten microscope glass slides are added to the Agarose-OTS prepared according to Example 15 and stirred at room temperature for 4 days. The slides are then washed in tap water for 5 minutes, and then air dried.

### Example 17

### Preparation of Agarose-dabco-C16

140.0 grams of Agarose-OTS prepared according to Example 15 is added to 1 mol equivalent (154.22 g) of dabco-C16 and stirred at room temperature for 4 days. The solvent completely evaporated and the surface hardened.

### Example 18

### Preparation of Microscope Glass-Agarose-dabco-C16

The Agarose-dabco-C16 prepared according to example 17 is dissolved in 600 ml H₂O. Ten microscope glass slides are added to the resulting solution for 1 day. The slides are then washed in tap water for 5 minutes and then air dried.

### Example 19

### Preparation of Agarose-OTS

200 mL of water is saturated with NaHCO₃. 5.0 grams of Argarose pure powder (MR=−0.13/−0.005) is treated with 1 mol equivalent of TsCl (5.33 g) and added to the saturated NaHCO₃ solution. The resulting mixture is stirred at room temperature for 1 day.

### Example 20

### Preparation of Agarose-OTS

100 mL of water is saturated with NaHCO₃. 5.0 grams of Argarose pure powder (MR=−0.13/−0.005) is treated with 1 mol equivalent of TsCl (5.33 g) and added to the saturated NaHCO₃ solution. The resulting mixture is stirred at room temperature for 1 day.

### Example 21

### Preparation of Ararose-dabco-C16

The liquid form of tosylated Agarose, Agarose-OTS prepared according to Example 19, is treated with 1 mol equivalent of dabco-C16 (JH23) and stirred at room temperature for 2 days.

### Example 22

### Preparation of Agarose-dabco-C16 (For spray bottle application)

The liquid form of tosylated Agarose, Agarose-OTS prepared according to Example 20, is treated with 1 mol equivalent of dabco-C16. 25 mL of water is added and the mixture is stirred at room temperature for 2 days. 100 mL of H₂O is added and the solution is stirred again at room temperature for 3 hours to dissolve all solid components.

### Example 23

### Preparation of Agarose-OTS

5.0 grams of Agarose pure powder (mix of 3.0 g MR=−0.02 and 2.0 g MR=−0.13+/−0.005) is added to 1 mol equivalent TsCl (5.34 g). The mixture is added to a saturated NaHCO₃ solution in 350 mL H₂O and stirred at room temperature for 4 days.

### Example 24

### Preparation of Agarose-dabco-C12

2.65 grams Agarose-OTS is treated with 1 mol equivalent of dabco-C12 (2.91 g) and stirred at room temperature for 1 day in 200 mL H₂O.

### Example 25

### Preparation of Microscope Glass-Agarose-dabco-C12 (washed)

Five microscope glass slides are placed in the Agarose-dabco-C12 prepared according to Example 26 and stirred at room temperature for 4 days. The slides are then washed in tap water for 5 minutes and air dried.

### Example 26

### Preparation of Microscope Glass-Agarose-dabco-C12 (unwashed)

Five microscope glass slides are placed in the Agarose-dabco-C12 prepared according to Example 26 and stirred at room temperature for 4 days. The slides are then air dried.

### Example 27

### Preparation of Glass Coverslips-Agarose-dabco-C12 (washed)

Five coverslips are placed in the Agarose-dabco-C12 prepared according to Example 26 and stirred at room temperature for 4 days. The coverslips are then washed in tap water for 5 minutes and air dried.

### Example 28

### Preparation of Glass Coverslips-Agarose-dabco-C12 (unwashed)

Five glass coverslips are placed in the Agarose-dabco-C12 prepared according to Example 26 and stirred at room temperature for 4 days. The coverslips are then air dried.

### Example 29

### Preparation of glycerol-(dabco-C12)ₘ where m is 2 or 3

A solution of glycerol is combined with p-toluenesulfonyl chloride in aqueous sodium bicarbonate. Dabco-C12 in ethanol is added to the solution. The mixture is stirred for 24 hours at ambient temperature.

### Example 30

### Preparation of paint containing glycerol-(dabco-C12)ₘ where m is 2 or 3

A solution of 10.0 g of glycerol-(dabco-C12) where m is 2 or 3 and 100 mL water is prepared. This solution is added to 907.2 g of paint and thoroughly mixed. The paint is then applied to a surface by spray or brush.

### Example 31

### Preparation of Latex Paint Containing Dabco

To a sample of 100 mL of a latex paint (Behr Premium Plus, #5340; Glidden Evermore Flat #EM9011; Minwax Polyacrylic Clear Satin; Minwax Polyacrylic Clear Gloss) is added 10 g, bis-1′,3′-(1-hexadecyl-1,4-diazoniabicyclo[2.2.2]octane-2′-propanol tetrachloride, and mixed in a blender for 1 min. The resultant mixture was applied to the appropriate surface, e.g., wood or polyurethane, and allowed to air dry.

We claim:

1. An antimicrobial composition comprising a carrier and a chemical compound having the following formula (1):
R¹—Y¹—X—Y²—R²
wherein:
X represents 1,4-diazoniabicyclo[2.2.2]octane;
Y¹ and Y² independently represent hydrocarbon chains comprising a minimum of 10 carbon atoms and a maximum of 24 carbon atoms;
R¹ and R² independently represent H, halo, or OR³;
R³ represents H or R⁴;
R⁴ represents —C(O)R⁵ or R⁶;
R⁵ represents H or a hydrocarbon group comprising a minimum of 1 carbon atom and a maximum of 4 carbon atoms; and
R⁶ represents a hydrocarbon group comprising a minimum of 1 carbon atom and a maximum of 4 carbon atoms.

2. An antimicrobial composition according to claim 1, wherein the composition is a pharmaceutical and the carrier is a pharmaceutically acceptable carrier suitable for topical administration.

3. An antimicrobial composition according to claim 1, wherein R¹ and R² represent H, Cl, or OH.

4. An antimicrobial composition according to claim 1, wherein R¹ and R² represent H.

5. An antimicrobial composition according to claim 1, wherein Y¹ and Y² comprise a hydrocarbon chain having 12 carbon atoms.

6. An antimicrobial composition according to claim 1, wherein Y¹ and Y² comprise a hydrocarbon chain having 16 carbon atoms.

7. An antimicrobial composition according to claim 1, wherein Y¹ comprises a hydrocarbon chain having 12 carbon atoms and Y² comprises a hydrocarbon chain having 16 carbon atoms.

8. An antimicrobial composition according to claim 1, wherein Y¹ and Y² each represent a mixture of hydrocarbon chains.

9. An antimicrobial composition according to claim 8, wherein at least 50% of the hydrocarbon chains represented by Y¹ comprise 12 carbon atoms and at least 50% of the hydrocarbon chains represented by Y² comprise 16 carbon atoms.

10. An antimicrobial composition according to claim 8, wherein at least 75% of the hydrocarbon chains represented by Y¹ comprise 12 carbon atoms and at least 75% of the hydrocarbon chains represented by Y² comprise 16 carbon atoms.

11. An antimicrobial composition according to claim 8, wherein at least 90% of the hydrocarbon chains represented by Y¹ comprise 12 carbon atoms and at least 90% of the hydrocarbon chains represented by Y² comprise 16 carbon atoms.

12. An antimicrobial composition according to claim 1, wherein the pharmaceutical composition is a gel.

13. An antimicrobial composition according to claim 1, wherein the pharmaceutical composition is a paste, lotion or spray.

14. An antimicrobial composition comprising a chemical compound having the following formula (2):
R¹—Y¹—X—Z—(X—Y²—R²)ₙ
wherein:
Z represents a polyol having more than one primary hydroxyl group wherein at least two of the primary hydroxyl groups have been replaced by R¹—Y¹—X or R²—Y²—X groups;
X represents 1,4-diazoniabicyclo[2.2.2]octane;
Y¹ and Y² independently represent hydrocarbon chains comprising a minimum of 10 carbon atoms and a maximum of 24 carbon atoms;
R¹ and R² independently represent H, halo, or OR³;
R³ represents H or R⁴;
R⁴ represents —C(O)R⁵ or R⁶;
R⁵ represents H or a hydrocarbon group comprising a minimum of 1 carbon atom and a maximum of 4 carbon atoms;
R⁶ represents a hydrocarbon group comprising a minimum of 1 carbon atom and a maximum of 4 carbon atoms; and
n represents any number up to m−1 wherein m represents the number of primary hydroxyl groups in the polyol.

15. An antimicrobial composition according to claim 14, wherein the polyol is an alkane polyol.

16. An antimicrobial composition according to claim 15, wherein the alkane polyol is glycerol, mannitol, ethylene glycol, or polyethylene glycol.

17. An antimicrobial composition according to claim 14, wherein the polyol is a carbohydrate.

18. An antimicrobial composition according to claim 14, wherein the polyol is a protein.

19. An antimicrobial composition according to claim 14, wherein the polyol is a gelling agent.

20. An antimicrobial composition according to claim 19, wherein the gelling agent is a polysaccharide.

21. An antimicrobial composition according to claim 20, wherein the polysaccharide is a gum, a pectin, agar, alginic acid or a salt thereof, or carrageenan.

22. An antimicrobial composition according to claim 19, wherein the gelling agent is a protein.

23. An antimicrobial composition according to claim 22, wherein the protein is a gelatin or collagen.

24. An antimicrobial composition according to claim 14, wherein Y¹ and Y² comprise a hydrocarbon group having 12, 14, 16, or 18 carbon atoms.

25. An antimicrobial composition according to claim 14, wherein Y¹ and Y² represents a mixture of hydrocarbon chains.

26. An antimicrobial composition according to claim 14, wherein the mixture of hydrocarbon chains comprises a hydrocarbon chain having 12 carbon atoms and a hydrocarbon chain having 16 carbon atoms.

27. An antimicrobial composition according to claim 14, wherein at least 25% of the hydrocarbon chains have 12 carbon atoms and at least 25% of the hydrocarbon chains have 16 carbon atoms.

28. An antimicrobial composition according to claim 14, wherein at least 75% of the hydrocarbon chains have 12 carbon atoms or 16 carbon atoms.

29. An antimicrobial composition according to claim 14, wherein at least 90% of the hydrocarbon chains have 12 carbon atoms or 16 carbon atoms.

30. An antimicrobial composition according to claim 14, wherein R¹ and R² independently represent H, Cl, or OH.

31. An antimicrobial composition according to claim 30, wherein R¹ and R² represent H.

32. An antimicrobial composition according to claim 14, wherein the composition is a paint.

33. An antimicrobial composition according to claim 14, wherein the composition is a fabric.

34. An antimicrobial composition according to claim 14, wherein the composition is a pharmaceutical composition.

35. An antimicrobial composition according to claim 34, wherein the pharmaceutical composition is a gel.

36. An antimicrobial composition according to claim 35, wherein the gel consists essentially of the chemical compound and water.

37. An antimicrobial composition according to claim 34, wherein the pharmaceutical composition is a paste, lotion or spray.

38. A method of making an antimicrobial composition comprising a chemical compound having the following formula (2):
R¹—Y¹—X—Z—(X—Y²—R²)ₙ
wherein:
Z represents a polyol having more than one primary hydroxyl group wherein at least two of the primary hydroxyl groups have been replaced by R¹—Y¹—X or R²—Y²—X groups;
X represents 1,4-diazoniabicyclo[2.2.2]octane;
Y¹ and Y² independently represent hydrocarbon chains comprising a minimum of 10 carbon atoms and a maximum of 24 carbon atoms;
R¹ and R² independently represent H, halo, or OR³;
R³ represents H or R⁴;
R⁴ represents —C(O)R⁵ or R⁶;
R⁵ represents H or a hydrocarbon group comprising a minimum of 1 carbon atom and a maximum of 4 carbon atoms;
R⁶ represents a hydrocarbon group comprising a minimum of 1 carbon atom and a maximum of 4 carbon atoms; and
n represents any number up to m−1 wherein m represents the number of primary hydroxyl groups in the polyol,
the method comprising:
(a) providing a solution of a polyol that has more than one primary hydroxyl group;
(b) converting at least two primary hydroxyl groups of the polyol to leaving groups; and
(c) adding R—Y-Q,
wherein R represents H, halo, or OR³;
Y represents a hydrocarbon chain comprising a minimum of 10 carbon atoms and a maximum of 24 carbon atoms; and
Q represents 1-azonia-4-azabicyclo[2.2.2]octane.

39. The method according to claim 38 wherein the leaving groups are sulfonate ester groups or halo groups.

40. The method according to claim 39 wherein the leaving groups are sulfonate ester groups selected from the group consisting of p-toluenesulfonate, benzenesulfonate, and methyl sulfonate.

41. The method according to embodiment 39 wherein the leaving groups are halo groups selected from the group consisting of chloro and bromo.
